(19) United States
(12) Patent Application Publication
(10) Pub. No.: US 2026/0232286 A1
(43) Pub. Date: Aug. 13, 2026

(54) Medical System Modification

(71) Applicant: Siemens Healthineers AG, Forchheim (DE)

(72) Inventors: Thomas Beck, Dormitz (DE); Philipp Höcht, Bavaria (DE)

(73) Assignee: Siemens Healthineers AG, Forchheim (DE)

(21) Appl. No.: 19/537,635

(22) Filed: Feb. 12, 2026

(30) Foreign Application Priority Data
Feb. 12, 2025 (DE) 10 2025 105 113.5

(51) Int. Cl. A61B 6/00; G16H 30/40; G16H 40/63

(52) U.S. Cl. CPC A61B 6/545; G16H 30/40; G16H 40/63

(57) ABSTRACT

A computer-implemented method for automatically modifying the configuration of a medical imaging system for an examination on an object, wherein the method includes: a) capturing at least one item of first context information, wherein the at least one item of first context information comprises at least one item of usage information about the usage situation of the medical imaging system, which usage situation is applicable for the examination; b) accessing a database and downloading first configuration data, which is associated with the at least one item of first context information, wherein the first configuration data comprises at least one selection of imaging protocols that is implementable during the examination on the object; and c) automatically modifying the configuration of the medical imaging system using the first configuration data.

Independent of the grammatical term usage, individuals with male, female, or other gender identities are included within the term.

### TECHNICAL FIELD

The present disclosure relates to a computer-implemented method for automatically modifying the configuration of a medical imaging system, to a computer program, and to a digital storage medium.

### BACKGROUND

Document EP 4 379 409 A1 relates to a method for operating a control unit and to a magnetic resonance tomography apparatus (MRT apparatus) comprising a control unit. The control unit is configured to drive, according to a specified measurement method, an MRT apparatus to perform, in accordance with the specification, a specified MRT measurement on the geometric imaging region of an object, and to receive an MRT dataset generated in the measurement. MRT examinations are each carried out in accordance with an associated specified MRT protocol. Further settings must be entered manually or altered manually.

To assist the user in selecting an MRT protocol, most MRT systems use standardized protocol trees. These offer the user a selection of MRT protocols with preset imaging parameters for different body parts and medical indications. An MRT protocol is understood to be a preset measurement sequence for an MRT device. For each patient examination, one or more MRT protocols are chosen manually from the protocol tree, and the further settings, for instance, imaging parameters, are entered or modified by a user, also referred to below as an operator. This is complex, time-consuming, and susceptible to errors. For example, the wrong protocol or wrong parameters may be chosen, or the information in the protocol tree may be out of date. A user must laboriously search for, enter, and check the settings individually in the protocol trees. Protocol trees are extensive and confusing, and therefore, an error, such as, for instance, selecting an incorrect measurement protocol, an out-of-date measurement protocol, or an incorrect parameter, can easily occur.

The success of a measurement depends mainly on the experience of the operator, because the manual entry of incorrect settings leads to flawed and unusable measurements. It is detrimental to the health of a patient if an incorrect measurement sequence or incorrect imaging parameters are used, for instance, because a measurement does not have the desired quality or a measurement has to be repeated.

### SUMMARY

Therefore, it can be considered an object of the present disclosure to provide a method for a medical imaging system that overcomes or reduces these and other disadvantages. It can be considered a further object of the disclosure to provide a method that improves the efficiency and/or the error susceptibility of a medical imaging system.

According to an aspect of the present disclosure, a computer-implemented method is provided for automatically modifying the configuration of a medical imaging system for an examination on an object, wherein the method comprises the following steps:
a) capturing at least one item of first context information, wherein the at least one item of first context information comprises at least one item of usage information about the usage situation of the medical imaging system, which usage situation is applicable for the examination;
b) accessing a database and downloading first configuration data, which is associated with the at least one item of first context information, wherein the first configuration data comprises at least one selection of imaging protocols that can be implemented during the examination on the object; and
c) automatically modifying the configuration of the medical imaging system using the first configuration data.

A method according to the disclosure can be used to capture a situational context of a medical imaging system and to modify the configuration of the imaging system accordingly. It is also disclosed how a medical imaging system can be optimally configured adaptively to a situational context. This can significantly improve both the efficiency and the error susceptibility during operation.

The following description of the exemplary aspects, which are explained in greater detail in conjunction with the figures, will clarify and elucidate the above-described properties, features, and advantages of this disclosure, and the manner in which they are achieved.

### BRIEF DESCRIPTION OF THE DRAWINGS

Aspects are described below with reference to the accompanying figures.

FIG. 1 shows a schematic flow diagram of a method according to an aspect of the present disclosure; and

FIG. 2 shows a schematic flow diagram of a method according to an aspect of the present disclosure.

### DETAILED DESCRIPTION

The background of the disclosure is that a large number of settings can be made at a modern medical imaging system, such as an MRT system, for example. This potentially leads to greater error susceptibility and a longer examination, because the users have to make these settings mostly manually. It would be possible to improve the work of the operating personnel by presetting and configuring the system according to the usage context. Such presets can also be referred to as “preferences”. Therefore, the disclosure captures-preferably automatically-the context of use, for instance, the user, the given usage situation, the patient, and/or the radiologist or doctor making the referral and/or diagnosis. A configuration, adaptation, and/or personalization of a medical imaging system is made by the method according to the disclosure, depending on a situational context. The method according to the disclosure can be implemented in particular automatically. In particular, the situational context can be identified automatically and the imaging system modified adaptively accordingly. This improves the quality of the examinations, shortens the examination time, and reduces the error susceptibility.

The term “configuration” comprises any type of setting or preset of a medical imaging system. The configuration can comprise, for example, a selection of protocols, in particular a protocol tree tailored to specific issues. In addition, the configuration can comprise a setting for the user interface, a preset for measurement parameters or protocols, attributes of image generation and image reconstruction, a pre-selection of the patient orientation, and/or a setting for image post-processing and data handling. This list is by no means exhaustive and is intended purely by way of example.

A “protocol” is understood to be, in particular, a measurement instruction for an imaging system, by means of which the system can perform a measurement on the object. A measurement is used to acquire typically one or more images of the object. There are different protocols for different body parts and indications. An indication is understood here to be a medical diagnosis or issue for which the patient is meant to be examined, e.g., a particular medical condition such as stroke, bone fracture, or torn ligament. An MRT protocol is understood to be a preset measurement sequence for an MRT device. Within a protocol, it is often still possible to set numerous imaging parameters.

A medical imaging system is a system that can be used for a medical examination, in particular for an imaging examination. A medical imaging system comprises at least one imaging device. The term is intended in the broad sense here and comprises all types of imaging devices, in particular larger imaging devices. A medical imaging device can be, in particular, a computed tomography device (CT device), an MRT device, a positron emission tomography device (PET device), an MR-PET device, a PET-CT device, a single photon emission computed tomography CT device (SPECT-CT device), or an SPECT device.

An object in the context of the present disclosure can be examined by such a medical imaging system as part of an examination. An object can be, for example, a human or person, or a body part of a human, for example, a patient or person under examination. The object can also be a non-living object, for instance, a phantom, which is meant to represent a patient or part of a patient, or a sample, for example, a tissue sample or a cultured sample.

For an examination, in particular, the object is introduced into an examination region of the imaging system, and one or more protocols are implemented. Image information or images are generated in the process, which are conveyed to a referring radiologist, for example. An examination on a patient typically lasts ½ to 1 h.

The method, according to the disclosure, is preferably repeated at regular intervals for a particular medical imaging system. According to an aspect, the method is performed before every examination. It can also be performed every day in the morning or whenever there is a change in user. This allows a dynamic context to be captured in addition to the static context.

The term “capturing” is intended in the broad sense in the present disclosure and includes all meanings of the term in general and technical usage. In particular, data and/or information can be captured by the method according to the disclosure. In particular, data and/or information can be retrieved from a data storage means, measured, acquired, and/or collected.

“First context information” comprises at least one item of information relating to a context of a medical imaging system. A context comprises all the data, information, settings, parameters, measurements, etc. that may be relevant to the use of the medical imaging system on the object. In particular, the context information can comprise usage information, i.e., information about the current type of use of the system, information about the current operator, the specific patient, and about the referring doctor or the radiologist. A context can be constant, at least constant over a certain time period, and/or can change over time. A change in a context can occur at different time periods, from a few minutes, through several hours, single days or a few days up to weeks, months, or years.

“Usage information” is an example of first context information. Usage information is information about a “usage situation” of a medical imaging system. A usage situation describes the type of use of the medical imaging system, for instance, whether it is being used in a clinical context or in research, or whether it is being used in a radiology clinic or in a (university) medical center. The usage situation can describe a spatial situation, for instance, the environment of a medical imaging system, for example, whether the medical imaging system is located in a radiology practice, in a hospital, or in a university medical center. There are also many imaging systems, however, that are used differently at different times, i.e., a temporal situation can change. It is often the case in university medical centers that a significantly different usage takes place from day to day; for example, Monday to Thursday, routine clinical practice; Friday to Sunday, usage for various studies and research applications. In addition, medical imaging systems are also sometimes leased by the hour or day to external affiliated doctors. Therefore, the usage information can also comprise a time-varying usage situation. The usage situation can change, for example, at different times of the day and night, on different days of the week, in different weeks or different months.

A usage situation can be derived, for example, from, in particular digitally, available calendars, a current operator or a current user, a current patient, a current examination, a current radiologist or current doctor, and/or a radiologist or doctor providing treatment. The method, according to the disclosure, can derive the given usage situation, preferably automatically.

“Configuration data” is, in particular, data that characterizes the “configuration” of the medical imaging system. Configuration data can comprise, for example, one or more of the following items of data or information:
a (pre)-selection of protocols, for example, a tailored protocol tree;
a (pre)-selection of imaging parameters;
a setting for a user interface, for example, the language or the display of additional input options or setting options. Setting options when using the medical imaging system as part of a scientific study can differ markedly from routine use as part of screening.
attributes or parameters of the image generation and/or the image reconstruction. The image generation and/or the image reconstruction can be modified depending on the situational system configuration. This is mostly achieved by using parameters or by selecting protocols, but more extensive modifications are also conceivable. For instance, additional or alternative scripts can be executed, which can be used to control and modify the process of image production.
a pre-selection of the patient orientation. The patient orientation is an example of how a typical parameterization depends on the situational context. For example, a medical imaging system can be used at times for breast-screening measurements and at other times mainly for orthopedic examinations. For times in which the breast screening is taking place, the default patient orientation setting might be “head first, prone,” for example, whereas preferably “feet first, supine” is more suitable for orthopedic examinations, mainly knee and ankle.
selection of prototypical measurement protocols and/or image post-editing algorithms. Especially in the research environment, there are a large number of prototypical methods that are unavailable in product software at a certain point in time. It is desirable to make the availability of these prototypical methods dependent on the situational configuration. This can ensure, for example, that routine patients are not measured during clinical imaging accidentally by methods not cleared for this purpose.
a setting for image post-processing and data handling. For example, image data can be routed in an automated manner to a dedicated DICOM node if the context suggests this. The quality of the examinations is improved, and the error susceptibility is reduced in this case.

This list is by no means exhaustive and is intended purely by way of example.

At least one item of first context information is associated with first configuration data. The first configuration data comprises at least item 1 in the list given above. It can also contain further items, however. The first configuration data relates to at least one usage situation, for example, at least one of the aforementioned usage situations. The first configuration data can depend on the environment of the medical imaging system. If, for example, the location is a university health center with a certain specialization, for instance, a certain body region, first configuration data can comprise all relevant measurement protocols for this specialization, or be oriented towards particular operators such as students, research doctors or assistant doctors, who differ from those in another environment, for instance, in a radiology practice. For example, university health centers also need, for instance, other prototypical methods and/or other measurement protocols than, for instance, a radiology practice or a hospital without a teaching and/or research mandate. These are illustrative examples of first configuration data.

Alternatively or additionally, the first configuration data can depend on a temporal usage situation. If the current time is a particular time of day, first configuration data can comprise a particular selection of protocols, which are employed by the user, for instance, an affiliated doctor, working at this time of day, or which are meant to be used in this usage situation.

This first configuration data is stored in a preferably digital database in correlation with at least one item of first context information. First, configuration data can be integrated into the database from different sources. A source can be a manufacturer, an operator, a user, a radiologist, and/or a doctor relating to a medical imaging system, whereby the corresponding first configuration data can be integrated into the database. The database can be updated at regular intervals, for example, by a manufacturer of the imaging system, an operator, a user, a radiologist, and/or a doctor. The database can be stored on a computer-readable medium or a computer. It can comprise a plurality of databases. A database can be stored in a network, for instance, a cloud.

The method according to the disclosure comprises accessing a database and downloading first configuration data, which is associated with the at least one item of first context information. As described above, the first configuration data comprises a selection of imaging protocols, for example, a modified protocol tree. A protocol tree comprises at least one protocol.

A multiplicity of imaging protocols, which can also be referred to as protocols or measurement protocols, are available for each medical imaging system. Usually, not all the imaging protocols are relevant for a particular item of first context information. Each particular item of first context information results in at least a partial limitation on the full number of imaging protocols. If the first context information comprises, for example, the usage situation “orthopedic practice,” the first configuration data contains only imaging protocols for examining orthopedic issues. On the other hand, imaging protocols for the heart, for example, are not relevant and can be disregarded. The method according to the disclosure accordingly provides a selection of imaging protocols that are relevant to the examination and can be implemented. For example, this can rule out any possibility of selecting an imaging protocol for the wrong body region. This reduces the error susceptibility. In addition, an operator does not have to go through all the imaging protocols that relate to irrelevant body regions, for example. This results in numerous advantages of the present disclosure. For example, the complexity and the time needed for the examination is reduced. This reduces the health-related stress for the patient. In addition, it relieves the strain on an operator and an object under examination, for example. In addition, the error susceptibility of an examination is reduced. For an object under examination, the examination is more pleasant, less of a strain and shorter, for instance, leading to reduced stress and to an improved examination and improved examination results. This improves the quality of the examination.

The term “modifying” is intended in the broad sense in the present disclosure and includes all meanings of the term in general and technical usage. In particular, modifying means that the configuration of a medical imaging system is modified in accordance with first configuration data. Modifying can mean that the first configuration data at least partially overwrites preset, e.g., factory-preset, configuration data of the medical imaging system.

“Automatically” means that an action, such as modifying the configuration of a medical imaging system, for example, takes place without the intervention of a user.

According to an aspect of the present disclosure, a computer program is provided. A computer program, which contains program code segments that cause a computer to perform a method for a floor-mounted medical device when the computer program is executed on the computer.

According to an aspect of the present disclosure, a digital storage medium is provided, on which is stored a computer program, which contains program code segments that cause a computer to perform a method for a floor-standing medical device when the computer program is executed on the computer.

According to an aspect of the present disclosure, a medical imaging system is provided, which is configured to perform the method according to the disclosure.

According to an aspect of the present disclosure, a computer-implemented method can comprise the further steps:
d) capturing at least one item of second context information, wherein the at least one item of second context information comprises, in particular, at least one item of operator information about a current user of the medical imaging system and/or at least one item of object-dependent information;
e) providing second configuration data, which is associated with the at least one item of second context information;
f) automatically modifying the configuration of the medical imaging system using the second configuration data, wherein the first configuration data is at least partially overwritten or supplemented.

Second context information comprises at least one item of information relating to a context of a medical imaging system. A context shall be understood to mean the same as described above. The second context information preferably relates to a situational, i.e., time-varying, context. Unlike first context information, the second context information does not comprise usage information about the applicable usage situation, but comprises at least one item of further context information, in particular a further item of the further context information described herein.

Second context information can comprise at least one fleet-specific parameter or item of information. Some owners have a multiplicity of medical imaging system, also known as a fleet, which are installed, for example, in different rooms or at different sites. Some owners have particular parameters that are meant to apply to all, or to particular, medical imaging systems regardless of the location of the medical imaging systems concerned.

Second context information can comprise a “system-specific” parameter or item of information. A system-specific parameter can comprise, for example, technical values such as information about the model of the imaging device, a magnetic field strength of the imaging device, or information about software installed on the imaging device.

The second context information can preferably comprise at least one radiologist-specific and/or patient-specific parameter or item of information. A radiologist-specific and/or patient-specific parameter can allow an association with a particular radiologist or patient.

In addition, the second context information can comprise at least one operator-specific parameter or item of information. An operator-specific parameter refers in particular to a particular operator or group of operators. An operator is, in particular, a person who operates the imaging device during the examination, for example, a radiographer.

Second context information can comprise at least one manufacturer parameter or item of information. Second context information can comprise at least one product-specific parameter or item of information. Second context information can comprise at least one country-specific parameter or item of information.

Operator information is information that comprises one of the above-described operator-specific parameters or items of information.

Object-dependent information is information that comprises at least one of the other parameters described above. Thus, object-dependent information comprises at least one item of information comprising at least one pre-configuration, a fleet-specific parameter, at least one system-specific parameter, at least one radiologist-specific and/or patient-specific parameter, at least one manufacturer parameter, at least one product-specific parameter, and/or at least one country-specific parameter.

At least one item of second context information can be associated with “second configuration data.” Second context information can comprise the aforementioned parameters or information. The second configuration data comprises at least one of the items 1 to 7 in the list given above. It can also contain further items, however.

There can be overlaps between first configuration data and second configuration data, because some information contained in the first configuration data may also be contained in the second configuration data, and vice versa. For example, a particular measurement protocol, associated with a usage situation as part of a protocol selection, can be first configuration data. The same or another measurement protocol, for instance, associated with a particular radiologist, can also be second configuration data. This is very advantageous because it allows customized configurations, i.e., configurations tailored to the operator, to be loaded very quickly. The system does not have to be completely reconfigured, but instead just the second configuration data is downloaded and replaces or supplements the first configuration data. According to an aspect, the first configuration data is at least partially overwritten by the second configuration data.

Second context information can be operator information, for example. Identification can be made by a login by means of a login portal, by means of deriving an operator through digitally processable information, for instance, (shift) schedules or overviews, by means of fingerprint matching, by means of iris matching, by means of a camera, or by means of reading an employee or personnel ID card, pass or another electronically analyzable identification feature. Second configuration data then contains the preferences of this specific operator or user for the pending examination. The second configuration data contains, for example, experience and background, for instance, an operator's experience with a particular software or software version, with a particular medical imaging system of a particular year of manufacture, or with a particular medical imaging system from another manufacturer, and the extent of this experience. The language skills or language preferences of an operator are also second configuration data. For an operator, based on the identification, a particular software version can be set in the medical imaging system if a particular operator has still not received training in another software version. In addition, some functions or measurement protocols can also be made available or not made available to a particular operator, depending on the examinations for which a particular operator or user is authorized and/or trained. This reduces the error susceptibility of an examination, improves the quality of an examination, and reduces the stress of an examination for a person to be examined.

The second context information can be radiologist-specific information, for example. A particular radiologist can prefer particular parameters, for instance, relating to measurement protocols, image contrasts, special measurements, particular measurement segments, particular measurement sequences, particular image orientations, particular image post-processing, particular data handling, and corresponding parameters in examinations. The specific parameters, the specific measurement protocol, the specific image contrast, the specific representation, etc., are then the corresponding second configuration data.

The second context information can comprise patient-specific information, for example, in particular, an identification of the particular patient (the object in the pending examination). The second configuration data is then defined, for instance, by the associated referring doctor or the radiologists making the diagnosis, who will receive the image data. The second context information can be transferred, for example, through an electronic interface from the referring doctor to the imaging system. Alternatively, for example, the patient can also provide an electronically readable medium (e.g., USB stick, storage medium) or a reference to information retrievable from the cloud (e.g., URL, 2D barcode, or the like). The context information provided in this way can contain the following information, for example:
a) selection of specific image contrasts or protocols that differ from the standard procedure;
b) preferences for image orientations (strictly parallel to the axes or aligned with the anatomy, e.g., along the AC-PC line (anterior commissure-posterior commissure) or transverse).

A manufacturer parameter as the second context information can be, for example, a particular measurement protocol, a particular function, or particular software, which comprises additional attributes to standard measurements and is offered only by a particular manufacturer. A manufacturer parameter can comprise attributes of a medical imaging system that are specific to a manufacturer.

In the case of a product-specific parameter as the second context information, a particular measurement protocol, for example, which comprises additional attributes to standard measurements, can comprise second configuration data.

In the case of country-specific parameters as the second context information, requirements, for example, legal requirements, that are relevant to, or necessary for, particular countries, can comprise the second configuration data.

The term “providing” is intended in the broad sense in the present disclosure and includes all meanings of the term in general and technical usage. In particular, providing can mean that second configuration data is retrieved from a data storage means, for example, from a hard disk or another data storage means, for instance, a remote memory or a cloud. In general, providing can also mean that second configuration data is entered in the form of information or data by a user or operator. Finally, providing can also include that second configuration data is generated in the form of information or data.

The terms “overwritten” or “supplemented” are intended in the broad sense in the present disclosure and include all meanings of the term in general and technical usage. In particular, the first configuration data can be changed at least partially. This can involve adding or supplementing information or data. It can involve replacing information or data with other information or data.

According to an aspect of the present disclosure, the second configuration data can be derived from the at least one item of first context information and/or from the at least one item of second context information, in particular by a self-learning algorithm.

In particular, a self-learning algorithm, also known as a learning algorithm, can be used in this aspect. Such an algorithm can learn solutions to problems that are too complicated to be described by rules, but for which there is a large amount of data that can serve as examples for the desired solution. Such an algorithm maps defined example data onto a mathematical model. The algorithm adapts the model such that it can generalize from the example data to new cases. This process is called training. After the training, the solution path found is stored in the model. It is not explicitly programmed. The trained model can make predictions for new data or produce recommendations and decisions. The self-learning algorithm can include a neural network, for example.

In the present disclosure, the self-learning algorithm can capture, for example, the operator-made selection of protocols, imaging parameters, image contrasts, image orientations, etc., and also capture the associated context automatically. In other words, both context information and the configuration data, which is set by the relevant operator, is captured for at least some examinations. The self-learning algorithm can thereby itself make the association of first context information with first configuration data, and, if applicable, of second context information with second configuration data, in particular automatically. This can be stored in the database. Then, if the same context arises again, for instance, because the same operator has logged into the console of the imaging device again, the associated configuration data is downloaded again. This can be preferred protocols and imaging parameters, or can be user-interface preferences, such as language, for example.

According to an aspect of the present disclosure, the second configuration data can be downloaded from a database.

It can be the same database as described above, in which first configuration data can be stored. It can be another database, in which case the description given above relating to the structure and/or attributes also applies here to this database.

According to an aspect of the present disclosure, the method can additionally comprise the steps: capturing changes to the configuration of the medical imaging system that are made, in particular by the user, for the examination, in particular changes to the user interface, changes to the at least one imaging protocol, and/or changes to the at least one imaging parameter; optionally requesting confirmation of whether the changes should be adopted in the second configuration data; automatically modifying the second configuration data on the basis of the changes made, in particular by the user; and conveying the modified second configuration data to the database.

A change can comprise adding and/or removing a configuration of a medical imaging system. The change can be made by a user. Such a change can be captured automatically by a method according to the disclosure, and forms the basis of automatic modification of second configuration data. This achieves ever improved modification and customization of the configuration data to suit the given context (user, radiologist, etc.).

Optionally, a request can be made before second configuration data is modified automatically. This is a check mechanism and makes it possible to reduce the error susceptibility to an incorrect change being adopted unchecked. An optional request can be made to a user, radiologist, or doctor. This may be a current user, radiologist or doctor, or a suitably authorized person who is located, for example, at a different site from the medical imaging system. An optional request can be made to another source, for example, to a cloud, a network, a database, a computer-readable medium, or a computer. A request can be made to a self-learning algorithm.

The modified second configuration data can be conveyed to a database. This may be a database as described above. It may be one of the above-described databases or another database, in which case the description given above relating to the structure and/or attributes also applies here to this database.

According to an aspect of the present disclosure, the method can additionally comprise the steps: storing changes to the configuration of the medical imaging system, which changes are made for the examination, over a certain time period, in particular a time period of a day or longer, in which a multiplicity of examinations are performed; calculating at least one item of relevance information, which is associated with each change made in the time period, wherein the relevance information is calculated from the frequency and optionally from the improvement potential of the change made; and prioritizing the changes made according to the at least one item of relevance information.

Changes that are made to a configuration of a medical imaging system can be stored. Storage can be made at any technically suitable location, for example, in one of the aforementioned databases and/or in a suitable storage medium. The storage time period is a correspondingly necessary and/or appropriate time period. The time period preferably covers a statistically relevant number of examinations, for example, between 20 and 1000, preferably 100 to 500. The time period can equal, for example, between 2 days and one year, preferably between one week and 6 months. By storing changes over a certain time period, the type and frequency of changes to a configuration of a medical imaging system can be documented and analyzed.

A frequency of a change made to a configuration can be used to capture or to calculate a relevance of a change. Relevance information can comprise a connection to various criteria relating to a change, for example, a weighting of changes. Other criteria, for instance, an improvement potential of a change made, can be used to further improve relevance information of a change.

Changes made can be assessed on the basis of at least one item of relevance information relating to at least one change made. Ranking, classification, and/or categorization of changes made may be performed. Prioritization of individual, a few, or a plurality of changes made may be performed. The term prioritizing means, in particular, that the importance of the changes made is established. It is thereby possible to capture which changes are also meant to be made to the first and/or second configuration data. The prioritized changes can be transferred to the database and are hence available for future implementations of the method or for future examinations. This can improve the configuration data and hence the situational preset for the system.

According to an aspect of the present disclosure, the method can additionally comprise the steps: anonymizing the changes to the configuration of the medical imaging system that are made for the examination; and conveying to the manufacturer of the medical imaging system the anonymized changes to the configuration.

Anonymizing is altering data or information in such a way that this data or information can no longer be associated with a particular origin, in particular a particular person, such as a radiologist, operator, or patient. Changes made can hence be used, investigated, and/or forwarded without infringing data protection laws. Anonymized changes can be forwarded and/or conveyed to numerous sources. A source can be a manufacturer, an operator, a user, a radiologist, and/or a doctor relating to a medical imaging system and/or a database. If anonymized changes are forwarded to the manufacturer, a manufacturer can ascertain, investigate and/or analyze changes and use them to improve and/or alter medical imaging systems.

According to an aspect of the present disclosure, the first configuration data can comprise one or more of the following items of configuration data: manufacturer-specific, product-specific, or country-specific parameters. These terms are to be understood as explained earlier.

According to an aspect of the present disclosure, the second configuration data can comprise a selection of imaging protocols, a pre-selection of at least one imaging parameter, at least one user-specific parameter, at least one system-specific parameter, at least one operator-specific parameter, and/or a configuration of a user interface.

A selection of imaging protocols for second configuration data can be understood to be similar to a selection of imaging protocols for first configuration data, and therefore, the description above relating to first configuration data is equally relevant, with the difference that it concerns second configuration data and not first configuration data. With regard to the terms imaging parameters, user-specific parameters, system-specific parameters, operator-specific parameters, and user interface, reference is made to the statements made elsewhere in this application, which are equally relevant.

According to an aspect of the present disclosure, the usage situation can comprise at least one spatial usage situation, at least one temporal usage situation, at least one current usage situation, and/or at least one environment situation.

With regard to the term usage situation, reference is made to the statements made elsewhere in this application.

An environment situation comprises at least one attribute of an environment of a medical imaging system. Such an attribute can comprise an attribute of a room in which a medical imaging system is integrated, or a limitation or structural attribute inside the room. Such an attribute can comprise at least one opening or at least one supply line, which can be limited, for instance, to a particular opening or particular region of a room.

According to an aspect of the present disclosure, the second configuration data can be part of the first configuration data, which is overwritten or supplemented.

As described above, an overlap is possible between at least one item of first context information and at least one item of second context information. An overlap is accordingly also possible between first configuration data and second configuration data. Thus second configuration data can also be part of first configuration data, and vice versa.

According to an aspect of the present disclosure, at least one object-dependent information can be a patient-specific or radiologist-specific parameter, wherein both parameters can be, for example, a specific protocol, a specific image contrast, a specific measurement, or a specific measurement method.

According to an aspect of the present disclosure, the medical imaging device can be, in particular, a CT device, an MRT device, a PET device, an MR-PET device, a PET-CT device, a SPECT device, or a SPECT-CT device.

According to an aspect of the present disclosure, a computer program is provided. A computer program according to the disclosure has, in particular, program code segments that model the method steps according to the disclosure when a computer program is executed on a computer.

It is thereby possible to define and repeatedly perform the method according to the disclosure, and to exercise control over disseminating the method according to the disclosure. The computer program is preferably configured such that a processing unit can perform the method steps according to the disclosure by means of the computer program. The program code segments can be loaded in particular into a memory of the processing unit, and typically can be executed by a processor of the processing unit with access to the memory. When the computer program, in particular the program code segments, is executed in the processing unit, typically all the aspects according to the disclosure of the described method can be implemented.

A computer program can be stored on a digital storage medium. The computer program is stored, for example, on a physical computer-readable medium and/or digitally as a data packet in a computer network. The computer program can constitute the physical computer-readable medium and/or the data packet in the computer network. Hence, the disclosure can also be based on the physical computer-readable storage medium and/or on the data packet in the computer network. The physical computer-readable storage medium can usually be connected directly to the processing unit, for instance, by inserting the physical computer-readable medium into a DVD drive or by plugging the same into a USB port, whereby the processing unit can have access, in particular read access, to the physical computer-readable medium. The data packet can preferably be retrieved from the computer network. The computer network can comprise the processing unit or be connected directly to the processing unit via a wide area network (WAN) connection and/or via a (wireless) local area network (WLAN or LAN) connection. For instance, the computer program may be held digitally on a cloud server at a storage location of the computer network, and be transferred by means of the WAN via the Internet and/or by means of the WLAN or LAN to the processing unit, in particular by opening a download link that points to the storage location of the computer program.

The disclosure is described and explained in greater detail below with reference to the exemplary aspects shown in the figures. The same reference signs are generally used in the following description of the figures to denote structures and units that remain substantially the same as in the first appearance of the structure or unit concerned.

All the aspects described herein can be combined with one another unless explicitly stated otherwise.

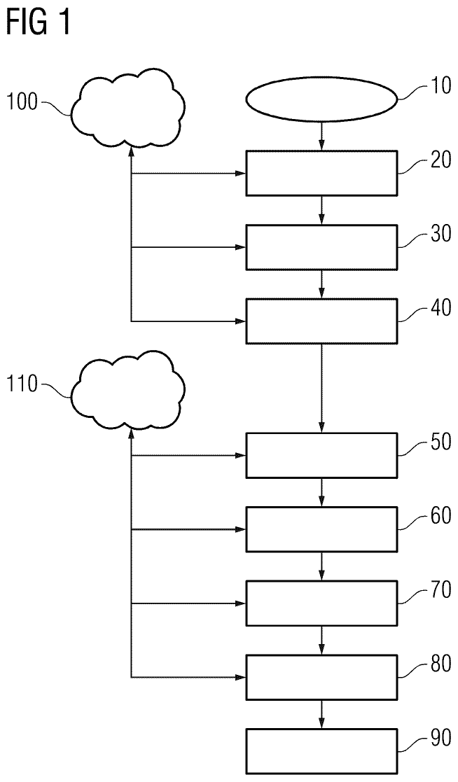

FIG. 1 shows a schematic flow diagram of a method according to an aspect of the present disclosure. Not all the steps shown are needed for all the aspects. Single or multiple steps can be omitted or not implemented. Single or multiple steps can be implemented at another point in time, i.e., the order of the steps can be varied. Further steps can additionally be implemented before or after individual described steps.

The method according to the disclosure shown in FIG. 1 relates to at least one item of second context information and to second configuration data.

A method according to the disclosure for automatically modifying the configuration of a medical imaging system for an examination on an object can comprise, in accordance with FIG. 1, a factory configuration 10 by a manufacturer of a medical imaging system. This is an initial configuration. In accordance with FIG. 1, the method according to the disclosure can comprise manufacturer 20, product-specific 30, country-specific 40, fleet-specific 50, system-specific 60, patient-specific/radiologist-specific 70, and/or operator-specific/user-specific 80 parameters. The factory configuration 10 and the aforementioned parameters 20, 30, 40, 50, 60, 70, 80 are examples of second context information, as described above. If more than one item of second context information is used in the method according to the disclosure, second context information can be taken into account simultaneously or in turn, where the order is not fixed or constrained. Therefore, the order shown in FIG. 1 is by way of example and not to be interpreted as restrictive. For each parameter, as second context information, second configuration data can be provided, as described above, which is associated with a second item of context information. Second configuration data for a particular item of second context information is described above, and therefore reference is made to these passages. In FIG. 1, the second configuration data is provided from two clouds 100, 110 by way of example. In FIG. 1, a manufacturer cloud 100 and an operator cloud 110 are shown by way of example. It is also possible for the Cloud to be a shared cloud or another source, such as a network, a database, a computer-readable medium, or a computer, for example.

In addition, second configuration data can be altered as described above. This means that there can be communication or feedback of the aforementioned parameters 20, 30, 40, 50, 60, 70, 80 to the at least one source 100, 110 of the second configuration data. The second configuration data can be at least partially altered, modified, or overwritten in the source 100, 110.

After the providing of second configuration data, which is associated with at least one item of second context information, there may be a modification to the system configuration 90. This modifying of the system configuration 90 can be performed automatically.

The method according to the disclosure shown in FIG. 1 can be repeated at least once, where more, less, and/or other second context information and/or second configuration data may be used in each repetition.

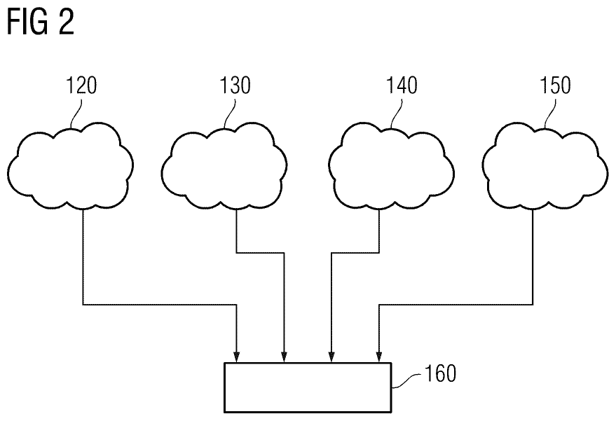

FIG. 2 shows a schematic flow diagram of a method according to an aspect of the present disclosure. Not all the steps shown are needed for all the aspects. Single or multiple steps can be omitted or not implemented. Single or multiple steps can be implemented at another point in time, i.e., the order of the steps can be varied. Further steps can additionally be implemented before or after individual described steps.

The method, according to the disclosure shown in FIG. 2, relates to at least one item of first context information and to at least one item of second context information. A usage situation 140 is first context information, as described above. An operator-specific/user-specific parameter 120, a patient-specific/radiologist-specific parameter 130, and a derived parameter 150 are examples of at least one item of second context information. A derived parameter 150 is second context information, which has been described above, but does not comprise, or does not only comprise, an operator-specific/user-specific parameter 120 or a patient-specific/radiologist-specific parameter 130. According to FIG. 2, the further items of second context information described above are derived parameters 150. On the basis of this first and/or second context information 120, 130, 140, 150, an association with first and/or second configuration data is made (not shown) and the situational system configuration 160 is modified. This modifying of the situational system configuration 160 can be performed automatically.

The disclosure relates to a computer-implemented method for automatically modifying the configuration of a medical imaging system for an examination on an object, wherein the method comprises the following steps: a) capturing at least one item of first context information, wherein the at least one item of first context information comprises at least one item of usage information about the usage situation of the medical imaging system, which usage situation is applicable for the examination; b) accessing a database and downloading first configuration data, which is associated with the at least one item of first context information, wherein the first configuration data comprises at least one selection of imaging protocols that can be implemented during the examination on the object; and c) automatically modifying the configuration of the medical imaging system using the first configuration data.

1. A computer-implemented method for automatically modifying a configuration of a medical imaging system for an examination on an object, the method comprising:
a) capturing at least one item of first context information, wherein the at least one item of first context information comprises at least one item of usage information about the usage situation of the medical imaging system, which usage situation is applicable for the examination;
b) accessing a database and downloading first configuration data, which is associated with the at least one item of first context information, wherein the first configuration data comprises at least one selection of imaging protocols that is implementable during the examination on the object; and
c) automatically modifying the configuration of the medical imaging system using the first configuration data.

2. The method as claimed in claim 1, further comprising:
d) capturing at least one item of second context information, wherein the at least one item of second context information comprises at least one item of operator information about a current user of the medical imaging system and/or at least one item of object-dependent information;
e) providing second configuration data, which is associated with the at least one item of second context information;
f) automatically modifying the configuration of the medical imaging system using the second configuration data, wherein the first configuration data is at least partially overwritten or supplemented.

3. The method as claimed in claim 2, wherein the second configuration data is derived from the at least one item of first context information and/or from the at least one item of second context information by a self-learning algorithm.

4. The method as claimed in claim 2, wherein the second configuration data is downloaded from a database.

5. The method as claimed in claim 4, further comprising:
capturing changes to the configuration of the medical imaging system made, changes to the user interface for the examination, changes to the at least one imaging protocol, and/or changes to the at least one imaging parameter;
optionally requesting confirmation of whether the changes should be adopted in the second configuration data;
automatically modifying the second configuration data based on the changes made by the user; and
conveying the modified second configuration data to the database.

6. The method as claimed in claim 5, further comprising:
storing changes to the configuration of the medical imaging system, which changes are made for the examination, over a certain time period, a time period of a day or longer, in which a plurality of examinations are performed;
calculating at least one item of relevance information, which is associated with each change made in the time period, wherein the relevance information is calculated from a frequency and from the improvement potential of the change made; and
prioritizing the changes made according to the at least one item of relevance information.

7. The method as claimed in claim 5, further comprising:
anonymizing the changes to the configuration of the medical imaging system that are made for the examination; and
conveying to a manufacturer of the medical imaging system the anonymized changes to the configuration.

8. The method as claimed in claim 1, wherein the first configuration data comprises one or more items of configuration data that is manufacturer-specific, product-specific, or country-specific parameters.

9. The method as claimed in claim 2, wherein the second configuration data comprises a selection of imaging protocols, a pre-selection of at least one imaging parameter, at least one user-specific parameter, at least one system-specific parameter, and/or a configuration of a user interface.

10. The method as claimed in claim 2, wherein the second configuration data is part of the first configuration data, which is overwritten or supplemented.

11. The method as claimed in claim 2, wherein the at least one item of object-dependent information is a patient-specific or radiologist-specific parameter, wherein both parameters are a specific protocol, a specific image contrast, a specific measurement, or a specific measurement method.

12. The method as claimed in claim 1, wherein the medical imaging system is a CT device, an MRT device, a PET device, an MR-PET device, a PET-CT device, a SPECT device, or a SPECT-CT device.

13. A medical imaging system, which is configured to perform the method as claimed in claim 1.

14. A digital storage medium, on which is stored a computer program, which includes program code segments that cause a computer to perform the method as claimed in claim 1 when the computer program is executed on the computer.
